(19) United States
(12) Patent Application Publication
(10) Pub. No.: US 2022/0226086 A1
(43) Pub. Date: Jul. 21, 2022

(54) COMPOSITIONS FOR CLEANSING REMOVABLE DENTAL PROSTHETICS

(71) Applicant: Armis Biopharma, Inc., Fort Collins, CO (US)

(72) Inventors: Scott Noblitt, Fort Collins, CO (US); Edwin Neas, Nunn, CO (US)

(21) Appl. No.: 17/596,518

(22) PCT Filed: Jun. 12, 2020
(86) PCT No.: PCT/US20/37616

§ 371 Date: Dec. 13, 2021

Related U.S. Application Data
(60) Provisional application No. 62/861,810, filed on Jun. 14, 2019.
(60) Provisional application No. 63/004,858, filed on Apr. 3, 2020.

(51) Int. Cl. A61C 17/00; A61K 8/38; A61K 8/22; A61K 8/362

(52) U.S. Cl. CPC A61C 17/036; A61K 8/362; A61K 8/22; A61K 8/38

(57) ABSTRACT

An aqueous cleansing composition for dental prosthetics and orthodontic devices, containing hydrogen peroxide, one or more carboxylic acids and one or more peracids, and methods for removing microorganisms and stains from dental prosthetics and other orthodontic devices using the cleansing compositions.

### FIELD

The present disclosure relates to a cleaning composition for eliminating microorganisms and removing stains from dental prosthetics and orthodontic devices.

### BACKGROUND

Removable dental prostheses (dentures) and other oral dental appliances require regular and frequent cleaning to prevent diseases such as stomatitis and pneumonia. Denture base acrylic resin and other polymers used for retainers and other removable orthodontic devices are easily colonized by oral endogenous bacteria in the mucosa underlying the denture and on the surfaces. This microbial reservoir can be responsible for denture-related stomatitis and aspiration pneumonia, a life threatening infection, especially in geriatric populations.

A need exists for better products for cleansing dental prostheses dental prostheses.

### SUMMARY

This patent document discloses a cleaning composition for eliminating microorganisms from dental prosthetics, which include for example dentures, caps, inlays, onlays, and bridges, as well as retainers and other removable orthodontic devices. In comparison with conventional products, the composition described herein is able to eliminate microorganisms such as bacteria, virus and fungus as well as removing biofilm, stain and other contaminants. The cleansing process can be completed in a short period of time and leaves no apparent damage to the dental prosthetics.

A first aspect of the disclosure provides an aqueous cleansing composition, containing: hydrogen peroxide in an amount ranging from about 125 ppm to about 25% by weight, one or more carboxylic acids in an amount ranging from about 150 ppm to about 30% by weight, and one or more peracids in an amount ranging from about 100 ppm to about 20% by weight, wherein at least one of the peracids has the same number of carbons as one of the carboxylic acids.

In one embodiment the composition further contains one or more C₁₋₅ alkyl hydro-peroxides ranging from about 1.25 ppm to about 5% by weight.

In another embodiment, the carboxylic acid is acetic acid. In one embodiment, the amount of acetic acid ranges from about 15% to about 20% by weight. In another embodiment, the amount of acetic acid ranges from about 150 ppm to about 20% by weight.

In another embodiment, the hydrogen peroxide ranges from about 125 ppm to about 20% by weight. In another embodiment, the hydrogen peroxide ranges from about 5% to about 20% by weight.

In a further embodiment, the one or more carboxylic acids range from about 150 ppm to 20% by weight. In a further embodiment, the one or more carboxylic acids range from about 5% to 20% by weight. In still a further embodiment, the one or more carboxylic acids are selected from C2-10 monocarboxylic acids, dicarboxylic acids, tricarboxylic acids, α-keto carboxylic acids, β-keto carboxylic acids, and mixtures thereof. In yet a further embodiment, the one or more carboxylic acids is acetic acid in an amount ranging from about 7% to about 20% of the total weight of the composition.

In a different embodiment, the one or more carboxylic acids further contains one or more additional acids selected from citric acid, succinic acid, glutaric acid, adipic acid, suberic acid, malonic acid, lactic acid, glycolic acid, oxalic acid, pyruvic acid, citramalic acid, acetoacetic acid, citraconic acid, maleic acid, and any mixture thereof.

In one embodiment, the peracid is peracetic acid. In another embodiment, the one or more peracids range in amount from about 100 ppm to about 12% of the total weight of the composition. In another embodiment, the one or more peracids range in amount from about 6% to about 12% of the total weight of the composition.

In another embodiment, the composition contains hydrogen peroxide in an amount ranging from about 5% to about 25% by weight, one or more carboxylic acids in an amount ranging from about 10% to about 30% by weight, and one or more peracids ranging from about 4% to about 20% by weight.

In yet another embodiment, the composition contains hydrogen peroxide in an amount ranging from about 5% to about 25% by weight, acetic acid in an amount ranging from about 5% to about 22% by weight, and peracetic acid in an amount ranging from about 0.25% to about 14% by weight.

In another embodiment, the aqueous cleansing composition contains hydrogen peroxide in an amount ranging from about 125 ppm to about 625 ppm, one or more carboxylic acids in an amount ranging from about 150 ppm to about 750 ppm, and one or more peracids in an amount ranging from about 100 ppm to about 500 ppm.

In another embodiment, the one or more carboxylic acids is acetic acid in an amount of from about 375 ppm to about 500 ppm. In a further embodiment, the one or more peracids in the composition is peracetic acid in an amount of from about 150 ppm to about 350 ppm.

In yet a further embodiment, the composition has a peracid concentration of between about 125 ppm and 400 ppm.

In still a further embodiment, the composition is diluted in water to a peracid concentration of between about 100 ppm and about 25,000 ppm.

In a further embodiment, the composition contains hydrogen peroxide in an amount ranging from about 125 to about 3 wt %, one or more carboxylic acids in an amount ranging from about 150 to about 3 wt %, and one or more peracids in an amount ranging from about 100 ppm to about 500 ppm, wherein at least one of the peracids has the same number of carbons as one of the carboxylic acids.

In one embodiment, the composition contains hydrogen peroxide in an amount ranging from about 55,000 ppm to about 120,000 ppm, one or more carboxylic acids in an amount ranging from about 70,000 ppm to about 120,000 ppm, and one or more peracids in an amount ranging from about 6,500 ppm to about 22,000 ppm, wherein at least one of the peracids has the same number of carbons as one of the carboxylic acids.

In another embodiment, the peracid concentration is between about 100 ppm and about 25,000 ppm. In a further embodiment, the composition contains hydrogen peroxide in an amount ranging from about 5,000 ppm to about 20,000 ppm, one or more carboxylic acids in an amount ranging from about 5,000 ppm to about 20,000 ppm, and one or more peracids in an amount ranging from about 100 ppm to about 300 ppm, wherein at least one of the peracids has the same number of carbons as one of the carboxylic acids.

The compositions of any of the aspects of the invention has a microbe kill time of less than five minutes.

In another embodiment the composition of any of the aspects of the invention contains one or more carboxylic acids selected from the group consisting of C2-10 monocarboxylic acids, dicarboxylic acids, tricarboxylic acids, α-keto carboxylic acids, β-keto carboxylic acids, and a mixture thereof. In a further embodiment, the composition of any aspects of the invention, contains one or more carboxylic acids and one or more additional acids selected from citric acid, succinic acid, glutaric acid, adipic acid, suberic acid, malonic acid, lactic acid, glycolic acid, oxalic acid, pyruvic acid, citramalic acid, acetoacetic acid, citraconic acid, maleic acid, and any mixture thereof.

In a further embodiment, the composition is in a form selected from a solution, gel or a foam. In yet a further embodiment, the composition of any aspects of the invention further contains a stabilizer. In still a further embodiment, the stabilizer is selected from etidronic acid, dipicolinic acid, and dipicolinic acid N-oxide. In a different embodiment, the composition of any of the aspects of the invention further contains an abrasive aid. In one embodiment, the abrasive aid is selected from calcium carbonate, a silica gel, silica precipitate, silicate, alumina, phosphate, phosphate salt, hydrated aluminum oxide, magnesium carbonate, and a combination thereof.

A second aspect of the invention provides a method for cleansing dental prosthetics and removable orthodontic devices, by contacting the dental prosthetic or removable orthodontic device with any of the compositions described herein. In one embodiment, the diluted composition described herein is contacted with the dental prosthetic or removable orthodontic device by dispensing a pressurized solution of the composition to on a surface of the dental prosthetic or removable orthodontic device. In another embodiment, the composition is contacted with the dental prosthetic or removable orthodontic device by soaking the surface of the dental prosthetic or removable orthodontic devices in a solution of one of the compositions described herein.

In a further embodiment of the method disclosed by this aspect, the composition contains hydrogen peroxide in an amount ranging from about 7% to about 25% by weight, acetic acid in an amount ranging from about 7% to about 22% by weight, and peracetic acid in an amount ranging from about 0.1% to about 14% by weight.

In a further embodiment of the disclosed method, the composition has a peracid concentration of between about 100 ppm and about 25,000 ppm.

In yet a further embodiment, the diluted composition used in the described method has: a hydrogen peroxide concentration ranging from about 5,000 ppm to about 20,000 ppm, a carboxylic acid concentration ranging from about 5,000 ppm to about 20,000 ppm, and a peracid concentration ranging from about 100 ppm to about 300 ppm.

In yet a further embodiment, the diluted composition used in the described method has: a hydrogen peroxide concentration ranging from about 125 ppm to about 20,000 ppm, a carboxylic acid concentration ranging from about 150 ppm to about 20,000 ppm, and a peracid concentration ranging from about 100 ppm to about 300 ppm. In yet a further embodiment, the dental prosthetic or removable orthodontic device is stained from contact with a food or beverage.

A fifth aspect of the present invention provides a solid product for cleansing dental prosthetics or removable orthodontic devices is provided, prepared by removing water from the aqueous compositions of the present invention. According to one embodiment, the composition is a solid powder in essentially the same relative ratios following removal of water. In another embodiment, the solid powder contains the magnesium salt of the carboxylic acid.

### BRIEF DESCRIPTION OF THE DRAWINGS

FIG. 1 is a photograph of a wine-stained denture and an unstained control before being cleaned with a composition according to the present invention; and

FIG. 2 is a photograph of the same wine-stained denture and control after cleaning.

### DETAILED DESCRIPTION

Various embodiments of this patent document provide an aqueous cleansing composition for eliminating bacteria, fungus, mycobacteria, and spores from dental prosthetics. The composition has been shown to prevent and/or destroy biofilms and subsequently kill all microorganisms present in biofilms on surfaces in short periods of time. An additional advantage over conventional method is that there is minimum impact on the appearance and durability of the dental prosthetics.

Some examples of the present disclosure will now be described more fully hereinafter with reference to the exemplified embodiments. Indeed, various aspects of the disclosure may be embodied in many different forms and should not be construed as limited to the examples set forth herein. Rather, these examples are provided so that this disclosure will be thorough and complete and will fully convey the scope of the disclosure to those skilled in the art.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “about” as used herein refers to the referenced numeric indication plus or minus 10% of that referenced numeric indication.

The term “C₁₋₅ alkyl” as used herein refers to an alkyl group, linear or branched, having 1, 2, 3, 4, or 5 carbons. Similarly, the term “C₁₋₁₀ alkyl” as used herein refers to an alkyl group, linear or branched, having carbons in the number ranging from 1 to 10.

The term “peracid” as used herein peracids refers to the oxidized form of a base organic acid (generally a carboxylic acid). Specifically the peracid has —(C═O)OOH group derived from —(C═O)OH of a carboxylic acid.

The term “alkyl hydroperoxide” refers to a structure based on that of a respective alkane wherein a hydrogen is replaced with OOH.

An aspect of this patent document provides an aqueous composition for cleansing dental prosthetics. The composition includes hydrogen peroxide ranging from about 125 ppm to about 25% by weight, one or more carboxylic acids ranging from about 150 ppm to about 30% by weight and one or more peracids ranging from about 100 ppm to about 20% by weight. The composition may also contain one or more C₁-5 alkyl hydroperoxides ranging from about 1.25 ppm to about 5% by weight in the composition. The composition can be in any suitable form, including for example, a solution, a foam or a gel. In some embodiments, the composition is an aqueous solution. Additional organic solvent can be added to improve the solubility of the components, the stability, and/or the efficacy of the composition.

Compositions according to the present invention may be prepared by conventional methods that are well-known to those of ordinary skill in the art. For example, 30 wt % hydrogen peroxide may be reacted with the desired carboxylic acid or a salt or anhydride thereof and allowed to equilibrate. Group II carboxylic acid salts may be used, and in particular magnesium salts, in order to prepare compositions that can be recovered as a solid powder by conventional means. Solid products will contain essentially the same ratio of ingredients as contained in the liquid product, but with the water removed, although the magnesium-based solids will have a higher level of organic acid, a lower level of peracid, and a significantly lower level of hydrogen peroxide.

As previously noted, the composition contains hydrogen peroxide. Hydrogen peroxide quantities disclosed herein are expressed as neat values, and are not quantities of hydrogen peroxide in solution. It should be noted that water in the aqueous composition includes water derived from the aqueous hydrogen peroxide used in manufacturing the composition. Any suitable aqueous hydrogen peroxide may be used in the composition, such as for example those containing up to 30% by weight, or up to 50% by weight of hydrogen peroxide. Higher or lower concentrations of hydrogen peroxide may be used in the composition, depending on the desired end product. Lower concentrations of hydrogen peroxide may be used with acid anhydrides. The ratio of starting materials is selected to provide the desired ratio of hydrogen peroxide, carboxylic acid and peracid in the final product. Tetraacetylethylenediamine (TAED) can also be reacted with hydrogen peroxide to produce the peracetic acid compositions of the present invention.

According to one embodiment, the composition contains hydrogen peroxide ranging from about 5% to about 25% by weight, acetic acid ranging from about 5% to about 22% by weight, and peracetic acid ranging from about 0.5% to about 14% by weight.

Individual components in the composition are adjusted to ensure the efficacy of eliminating undesirable microorganisms and maintaining the quality of the dental prosthetics. In some embodiments, the hydrogen peroxide ranges from about 5% to about 25%, from about 7% to about 20%, from about 7% to about 18%, or from about 7% to about 12%, all by weight.

The carboxylic acids can be C₂₋₁₀ monocarboxylic acid, dicarboxylic acid, tricarboxylic acid, α-keto carboxylic acid, β-keto carboxylic, or a mixture thereof. Non-limiting examples of the carboxylic acids include acetic acid, propionic acid, citric acid, succinic acid, glutaric acid, adipic acid, suberic acid, malonic acid, lactic acid, glycolic acid, oxalic acid, pyruvic acid, citramalic acid, acetoacetic acid, citraconic acid, maleic acid, and a mixture thereof.

In some embodiments, the one or more carboxylic acids, individually or totally, range from about 5% to about 30%, from about 5% to about 25%, from about 5% to about 22%, from about 5% to about 20%, from about 7% to about 18%, or from about 10% to 30% all by weight in the composition.

In some embodiments, the composition contains acetic acid ranging from about 5% to about 25%, from about 5% to about 22%, from about 5% to about 20%, from about 4% to about 20%, or about 7% to about 19% of the total weight of the composition. In some embodiments, the composition further contains one or more additional acids selected from, citric acid, succinic acid, glutaric acid, adipic acid, suberic acid, malonic acid, lactic acid, glycolic acid, oxalic acid, pyruvic acid, citramalic acid, acetoacetic acid, citraconic acid, maleic acid and any mixture thereof.

In some embodiments, the one or more additional acids, individually and independently if present, range from about 0.001% to about 0.2%, from about 0.001% to about 0.15%, from about 0.002% to about 0.15%, from about 0.02% to about 0.15%, from about 0.02% to about 0.10%, from about 0.05% to about 0.1%, from about 0.06% to about 0.08%, from about 0.07% to about 0.08%, from about 0.002% to about 0.02%, from about 0.003% to about 0.015%, from about 0.004% to about 0.015%, from about 0.010% to about 0.02%, from about 0.010% to about 0.015%, from about 0.010% to about 0.013%, from about 0.011% to about 0.013%, from about 0.003% to about 0.02%, from about 0.003% to about 0.01%, from about 0.003% to about 0.008%, or from about 0.004% to about 0.006% by weight.

The amount of peracid is adjusted in a suitable range to achieve a desirable effect in combination of the carboxylic acid(s). In some embodiments, the peracid ranges from about 0.1% to about 20%, from about 0.25% to about 14%, from about 0.5% to about 12%, from about 0.75% to about 10%, or from about 1% to about 10%, all by weight of the composition.

In some embodiments, the peracid has the same number of carbons as one of the carboxylic acids. In some embodiments, the peracid is peracetic acid. In some embodiments, the composition includes acetic acid and peracetic acid.

The composition also contains C₁-5 alkyl hydroperoxide, which can be for example, methyl hydroperoxide, ethyl hydroperoxide, propyl hydroperoxide, or any combination thereof. The C₁₋₅ alkyl hydroperoxide ranges from about from about 1.25 ppm to about 5%, from about 0.05% to about 5%, from about 0.1% to about 5%, from about 0.1% to about 2%, from about 0.1% to about 1%, from about 0.2% to about 2%, from about 0.2% to about 1%, from about 0.3% to about 1%, from about 0.2% to about 0.8%, from about 0.3% to about 0.6%, or from about 0.4% to about 0.6% by weight.

In some embodiments, the composition also includes a geminal dihydroperoxy substituted C₃₋₁₀ carboxylic acid ranging from about 0.05% to about 2% by weight. Non-limiting examples of the geminal dihydroperoxy substituted C₃₋₁₀ carboxylic acid include 3,3-bis(hydroper-oxybutanoic acid and 3,3-bis(hydroperoxy)pentanoic acid. Additional exemplary ranges of the acid include from about 0.05% to about 1%, from about 0.05% to about 0.5%, from about 0.05% to about 0.8%, from about 0.05% to about 0.5%, from about 0.1% to about 0.3%, or from about 0.1% to about 0.2% by weight. In some embodiments, the composition also includes a geminal dihydroperoxy substituted C₃₋₁₀ alkyl. Non-limiting examples of the geminal dihydroperoxy substituted C₃₋₁₀ alkyl include 2,2-bis(hydroperoxy)propane, 2,2-bis(hydroperoxy) butane, and 2,2-bis(hydroperoxy)pentane. In some embodiments the germinal dihydroperoxy substituted C₃₋₁₀ alkyl ranges from about 0.005% to about 0.2%, from about 0.005% to about 0.1%, from about 0.01% to about 0.2%, from about 0.01% to about 0.1%, from about 0.01% to about 0.05%, from about 0.01% to about 0.03%, or from about 0.02% to about 0.03% by weight.

In some embodiments, the composition includes a stabilizer. Any suitable stabilizer may be used, such as for example, an acid such as etidronic acid, dipicolinic acid, or dipicolinic acid N-oxide. The stabilizer may be present in an amount ranging from about 0.003% to about 0.05%, from about 0.003% to about 0.03%, from about 0.005% to about 0.05%, from about 0.008% to about 0.05%, from about 0.008% to about 0.02%, from about 0.008% to about 0.01%, or from about 0.008% to about 0.015% by weight.

In some embodiments, the composition includes an abrasive aid that can contribute to the cleansing capability of the composition, without causing damage to the dental prosthetic. Any suitable abrasive aid may be used, such as for example, calcium carbonate, a silica gel, silica precipitate, silicate, alumina, phosphate, phosphate salt, hydrated aluminum oxide, magnesium carbonate, or a combination thereof. Specific examples include dicalcium orthophosphate dihydrate, calcium pyrophosphate, tricalcium phosphate, hydrated alumina, beta calcium pyrophosphate, and resinous abrasive materials.

Another aspect of the document includes a method of cleansing dental prosthetics. The method is effective for removing or destroying sludge, biofilm, bacteria, stain and other contaminants from the dental prosthetics. The method generally includes contacting the cleansing composition with the dental prosthetics. When used as a solution, the peracid concentration of the composition can be adjusted depending on the pathogens or contaminants to be removed. Typical peracid concentrations of solutions diluted in water range between about 50 and about 25,000 ppm. According to one embodiment, the peracid concentration is between about 100 ppm and about 23,000 ppm.

According to one embodiment, the peracid concentration is between about 6,500 ppm and about 23,000 ppm. According to one embodiment, the peracid concentration is between about 6,500 ppm and about 11,500 ppm. According to one embodiment, the peracid concentration is between about 13,000 ppm and about 22,000 ppm.

According to one embodiment, the peracid concentration is between about 1500 ppm and about 15,000 ppm. According to one embodiment, the peracid concentration is between about 2500 ppm and about 10,000 ppm. According to another embodiment, the peracid concentration is between about 3000 and about 8000 ppm.

According to one embodiment, the peracid concentration is between about 50 ppm and about 3,000 ppm. According to one embodiment, the peracid concentration is between about 70 ppm and about 2,500 ppm. According to one embodiment, the peracid concentration is between about 90 ppm and about 1,500 ppm. According to one embodiment, the peracid concentration is between about 100 ppm and about 1,000 ppm.

According to one embodiment, the peracid concentration is less than 5000 ppm. According to one embodiment, the peracid concentration is less than 2500 ppm. According to one embodiment, the peracid concentration is less than 1000 ppm. According to one embodiment, the peracid concentration is less than 750 ppm.

The composition may be highly diluted, such that the peracid concentration of the diluted composition is less than 500 ppm. According to one embodiment, the peracid concentration is between about 100 ppm and about 500 ppm. According to one embodiment, the peracid concentration is between about 100 ppm and about 300 ppm. According to one embodiment, the peracid concentration is between about 125 ppm and about 400 ppm. According to one embodiment, the peracid concentration is between about 150 ppm and about 300 ppm. According to one embodiment, the peracid concentration is between about 150 ppm and about 350 ppm.

According to one embodiment, the peracid concentration is less than 500 ppm. According to one embodiment, the peracid concentration is less than 250 ppm. According to one embodiment, the peracid concentration is less than 100 ppm.

In one embodiment, the highly diluted composition may contain from about 5,000 ppm to about 20,000 ppm of hydrogen peroxide. According to one embodiment, the highly diluted composition may contain from about 5,000 ppm to about 20,000 ppm of the carboxylic acid.

In one embodiment, the diluted cleansing composition may contain hydrogen peroxide in an amount ranging from about 5,000 ppm to about 20,000 ppm, one or more carboxylic acids in an amount ranging from about 5,000 ppm to about 20,000 ppm, and one or more peracids in an amount ranging from about 100 ppm to about 300 ppm

In another embodiment, the aqueous cleansing composition, contains hydrogen peroxide in an amount ranging from about 125 ppm to about 625 ppm, one or more carboxylic acids in an amount ranging from about 150 ppm to about 750 ppm, and one or more peracids in an amount ranging from about 100 ppm to about 500 ppm.

In one embodiment, the concentration of the carboxylic acid in the dilute composition, may be below 20000 ppm. According to one embodiment, the carboxylic acid concentration may be below 15000 ppm. According to one embodiment, the carboxylic acid concentration may be below 1000 ppm. According to one embodiment, the carboxylic acid concentration is between about 5000 ppm and about 20000 ppm.

According to one embodiment, the carboxylic acid concentration is between about 250 ppm and about 750 ppm. According to one embodiment, the carboxylic acid concentration is between about 300 ppm and about 650 ppm. According to one embodiment, the carboxylic acid concentration is between about 350 ppm and about 600 ppm.

According to one embodiment, the carboxylic acid concentration is between about 300 ppm and about 550 ppm. According to one embodiment, the carboxylic acid concentration is less than 750 ppm. According to one embodiment, the carboxylic acid concentration is less than 650 ppm. According to one embodiment, the carboxylic acid concentration is less than 500 ppm.

In one embodiment, the concentration of the hydrogen peroxide may be below 700 ppm. According to one embodiment, the hydrogen peroxide concentration is between about 125 ppm and about 625 ppm. According to one embodiment, the hydrogen peroxide concentration is between about 135 ppm and about 550 ppm. According to one embodiment, the hydrogen peroxide concentration is between about 145 ppm and about 450 ppm. According to one embodiment, the hydrogen peroxide concentration is between about 150 ppm and about 350 ppm.

According to one embodiment, the hydrogen peroxide concentration is less than 625 ppm. According to one embodiment, the hydrogen peroxide concentration is less than 550 ppm. According to one embodiment, the hydrogen peroxide concentration is less than 400 ppm.

The diluted composition may have a microbe kill time of less than about less than about 5 minutes.

In one embodiment, the dilute composition contains one or more carboxylic acids selected from C2-10 monocarboxylic acid, dicarboxylic acid, tricarboxylic acid, α-keto carboxylic acid, β-keto carboxylic acid, and a mixture thereof. In another embodiment, the dilute composition further contains one or more additional acids selected from propionic acid, citric acid, succinic acid, glutaric acid, adipic acid, suberic acid, malonic acid, lactic acid, glycolic acid, oxalic acid, pyruvic acid, citramalic acid, acetoacetic acid, citraconic acid, maleic acid, and any mixture thereof. In one embodiment, the carboxylic acid may include acetic acid in an amount of from about 375 ppm to about 500 ppm.

In one embodiment, the peracid in the composition is peracetic acid, in an amount of from about 150 ppm to about 350 ppm.

In another embodiment, the dilute composition further contains one or more C1-5 alkyl hydro-peroxides in an amount ranging from about 1.25 ppm to about 125 ppm.

Another aspect of the invention includes a method for cleansing dental prosthetics and removable orthodontic devices, including contacting the dental prosthetic or removable orthodontic device with the dilute composition.

In some embodiments, the time of contact is less than about 45 minutes, less than about 30 minutes, less than about 20 minutes, less than about 15 minutes, less than about 10 minutes, less than about 5 minutes, or less than about 1 minute. The contact time can be a length of time effective to remove biofilm and/or kill microorganisms. The contact can be accomplished by spraying a solution of the composition to the dental prosthetics, for example with a Waterpik dispensing the solution at high pressure. Alternatively, the dental prosthetics can be soaked in the solution. If necessary, a step of brushing or scrubbing the dental prosthetics surface can be added. Sonication is also an optional step to facilitate the cleansing process. Therefore, the ability of the composition described herein to disinfect dental prosthetics by removing biofilm and killing microorganisms such as yeast, virus and bacteria effectively reduces a user's risk to diseases and conditions including for example gingivitis, stomatitis and or Cheilitis.

### EXAMPLES

### Example 1

Treatment of contaminated dentures with a composition of the present invention, identified as Formula I, using a water flosser was studied. Formula I was an aqueous solution containing 19.6 wt % hydrogen peroxide, 16.8 wt % acetic acid, 10.3 wt % peracetic acid, and less than 0.3 wt % of other carboxylic acids. All extracted microbes were identified (Table 1) and the mean kill for 4 dentures was 99.9992 percent after 15-minute treatment (Tables 2A (control) and 2B (treatment with Formula I).

TABLE 1
Extracted Bacteria From Patient Dentures

| DENTURE ONE | DENTURE TWO | DENTURE THREE | DENTURE FOUR |
|---|---|---|---|
| Streptococcus salivarius | Lactobacillus rhamnosus | Actinomyces oris | Actinomyces oris |
| Streptococcus oralis | Rothia dentocariosa | Candida glabrata | Candida albicans |
| Streptococcus parasanguinis | Rothia mucilaginosa | Lactobacillus parabuchneri | Candida glabrata |
| Actinomyces oris | Staphylococcus epidermidis | Lactobacillus rhamnosus | Lactobacillus gasseri |
| Rothia mucilaginosa | Staphylococcus hominis | Rothia mucilaginosa | Lactobacillus rhamnosus |
| Staphylococcus warneri | Streptococcus pneumoniae | Streptococcus mutans | Presumptive Corynebacterium kroppendstedtii |
| | Streptococcus salivarius | Streptococcus mutans | Propionibacterium freundenreichii |
| | Streptococcus sanguinis | Streptococcus oralis | Staphylococcus hominis |
| | Streptococcus species | Streptococcus salivarius | Streptococcus species |
| | Veillonella dispar | Candida glabrata | Candida albicans |

TABLE 2A
Efficacy in the Treatment of Contaminated Dentures
Enumeration Data (CFU/Denture)
Control

| | Aerobic | Anaerobic | Yeast | Overall¹ |
|---|---|---|---|---|
| Rep 1 | 1.7E+09 | 4.2E+09 | 1.3E+05 | 5.9E+09 |
| Rep 2 | 2.0E+06 | 1.7E+06 | 2.0E+02 | 3.7E+06 |
| Rep 3 | 3.2E+07 | 2.3E+05 | 2.4E+05 | 3.2E+07 |
| Rep 4 | 3.2E+06 | 4.6E+06 | 8.2E+05 | 8.6E+06 |
| Geom Mean | 2.43E+07 | 9.32E+06 | 4.76E+04 | 4.97E+07 |

TABLE 2B
Efficacy in the Treatment of Contaminated Dentures (Cont'd)
Enumeration Data (CFU/Denture)
8000 ppm Treatment

| | | | | | Overall |
|---|---|---|---|---|---|
| | | | | | percent |
| | Aerobic | Anaerobic | Yeast | Overall | kill² |
| Rep 1 | 2.0E+03 | 1.0E+00 | 1.0E+00 | 2.0E+03 | 99.99997 |
| Rep 2 | 1.0E+00 | 1.0E+00 | 1.0E+00 | 3.0E+00 | 99.99992 |
| Rep 3 | 2.0E+03 | 1.0E+00 | 1.0E+00 | 2.0E+03 | 99.99383 |
| Rep 4 | 1.0E+00 | 1.0E+00 | 2.0E+03 | 2.0E+03 | 99.97677 |
| Geom Mean | 4.47E+01 | 1.00E+00 | 6.69E+00 | 3.94E+02 | 99.99921 |

In addition, denture treatment with the composition of the present invention in comparison with marketed commercial cleaners clearly demonstrated that the current commercials cleaners have lower bacterial kill relative to the present invention (Table 3 and Table 4).

TABLE 3
Comparison With Commercial Cleaners

| | | Aerobic | | Anaerobic | |
|---|---|---|---|---|---|
| | Treatment | Denture | Log | Denture | Log |
| | Time | CFU | Change | CFU | Change |
| Control | N/A | 4.7E+05 | N/A | 1.1E+06 | N/A |
| Fixodent Kill | 15 min | 7.4E+02 | −2.8 | 1.2E+03 | −3.0 |
| Formula 1 | 15 min | 1.0E+01 | −4.7 | 1.0E+00 | −6.0 |

TABLE 4
Comparison With Commercial Cleaners

| | | Aerobic | | Anaerobic | | Yeast | |
|---|---|---|---|---|---|---|---|
| | Treatment | Denture | Log | Denture | Log | Denture | Log |
| | Time | CPU | Change | CFU | Change | CFU | Change |
| Control | N/A | 4.4E+06 | N/A | 3.2E+07 | N/A | 2.8E+04 | N/A |
| Polident Kill | 15 min | 3.2E+05 | −1.4 | 1.9E+06 | −1.4 | 3.5E+05 | 1.1 |
| Formula I | 15 min | 2.0E+02 | −4.3 | 2.0E+02 | −4.2 | 4.0E+03 | −0.8 |
| Control | N/A | 5.4E+08 | N/A | 3.2E+08 | N/A | 1.0E+06 | N/A |
| Efferdent Kill | 15 min | 1.0E+06 | −2.7 | 6.6E+05 | −2.8 | 4.4E+04 | −1.4 |
| Formula I | 15 min | 2.0E+03 | −5.4 | 1.0E+01 | −8.6 | 1.0E+00 | −6.0 |

To examine any detrimental effect on dentures by the composition, a single denture was cut in half. One-half was exposed to a water control and the other half exposed to 8000 ppm of a solution of Formula I for 15 minutes 32 times. No visible difference was observed after the treatment between the two halves.

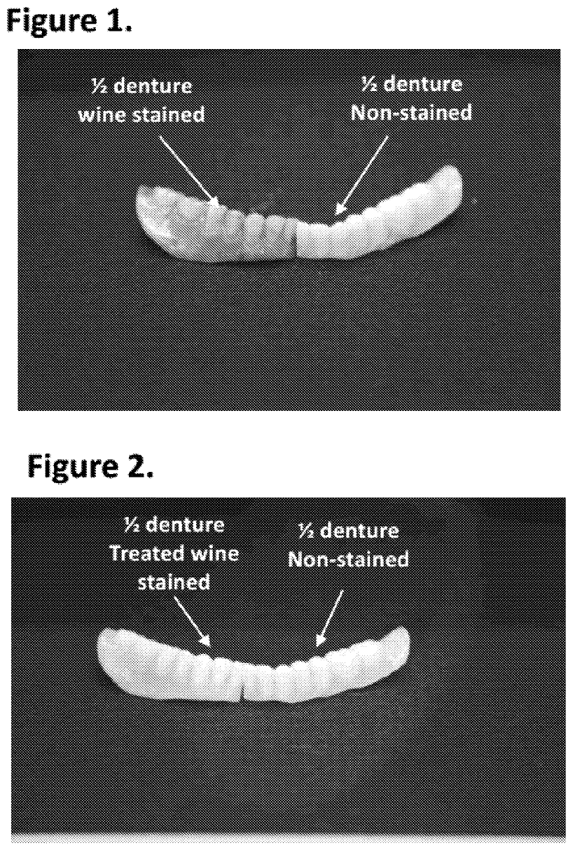

A de-staining study was initiated to determine if the composition would remove wine-stained dentures. A clean denture was cut in half and one half was wine stained. The wine-stained half was exposed to a solution of Formula I for 15 minutes 10 times with no apparent damage to the denture and removed all stain. The results are depicted in FIGS. 1 and 2.

### Example 2

The time to kill pathogens using the compositions described in Table 5 was studied. The time to kill selected microbes was determined and recorded in Table 6.

TABLE 5
Composition

| | Hydrogen | Acetic | Peracetic |
|---|---|---|---|
| | Peroxide | Acid | Acid |
| Pathogen | (ppm) | (ppm) | (ppm) |
| Acinetobacter spp. | 135 | 129 | 73 |
| Aspergillus spp. | 4720 | 4510 | 2560 |
| Candida spp. | 538 | 515 | 292 |
| Candida auris | 1750 | 1670 | 948 |
| Clostridium difficile | 4720 | 4510 | 2560 |
| Enterobacteriaceae | 1350 | 1290 | 731 |
| Enterococcus spp. | 135 | 129 | 73 |
| Mycobacterium tuberculosis | 1350 | 1290 | 731 |
| complex | | | |
| Non-tuberculosis mycobacteria | 673 | 643 | 365 |
| spp. | | | |
| Pseudomonas spp. | 269 | 257 | 146 |
| Staphylococcus aureus | 404 | 386 | 219 |
| Streptococcus pyogenes | 404 | 386 | 219 |

TABLE 6
Time to Kill Microbes

| GAIN Act | Veriox ® Efficacy | Log 10 | Time to Kill |
|---|---|---|---|
| Pathogen List | Test Pathogen | Kill | @ppm peracid |
| Acinetobacter spp. | A. baumannii | ≥6 | ≤15 sec @73 ppm |
| Aspergillus spp. | A. genus | ≥5.3 | ≤10 min@2560 ppm |
| Candida spp. | C. albicans | ≥5.4 | ≤10 sec @292 ppm |
| Candida auris\hat{ } | C. Auris | ≥7 | ≤5 min @948 ppm |
| Clostridium difficile | C. difficile | ≥6 | ≤5 min @2560 ppm |
| Enterobacteriaceae | CRE ® (Klebsiella pneumonia) | ≥5.4 | ≤1 min @731 ppm |
| Enterococcus spp. | E. faecalis | ≥6 | ≤1 min @73 ppm |
| Mycobacterium tuberculosis complex | M. tuberculosis | ≥8 | ≤5 min @731 ppm |
| Non-tuberculosis mycobacteria spp. | M. chelone | ≥8 | ≤1 min @365 ppm |
| Pseudomonas spp. | P. aeruginosa | ≥6 | ≤10 sec@146 ppm |
| Staphylococcus aureus | MRSA† | ≥6 | ≤20 sec @219 ppm |
| Streptococcus pyogenes | S. pyogenes | ≥6 | ≤15 sec @219 ppm |
| Influenza pandemic virus\hat{ } | H1N1p | ≥6 | ≤5 min |
| Enterovirus 71\hat{ } | EV71 | ≥6 | ≤10 min |

*Carbopenem-resistant Enterobacteriaceae.
†Methicillin-resistant Staphylococcus aureus.
\hat{ }Not GAIN Act pathogens

Many modifications and other examples of the disclosure set forth herein will come to mind to those skilled in the art to which this disclosure pertains, having the benefit of the teachings presented in the foregoing descriptions and the associated drawings. Therefore, it is to be understood that the disclosure is not to be limited to the specific examples disclosed and that modifications and other embodiments are intended to be included within the scope of the appended claims.

Moreover, although the foregoing descriptions and the associated embodiments describe aspects of the disclosure in the context of certain example combinations of structural elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1. An aqueous cleansing composition, comprising:
hydrogen peroxide in an amount ranging from about 125 ppm to about 25% by weight,
one or more carboxylic acids in an amount ranging from about 150 ppm to about 30% by weight, and
one or more peracids in an amount ranging from about 100 ppm to about 20% by weight,
wherein at least one of the peracids has the same number of carbons as one of the carboxylic acids.

2. The cleansing composition of claim 1, further comprising one or more C₁₋₅ alkyl hydro-peroxides ranging from about 1.25 ppm to about 5% by weight.

3. The composition of claim 1, wherein the hydrogen peroxide ranges from about 125 ppm to about 20% by weight.

4. The composition of claim 1, wherein the hydrogen peroxide ranges from about 10% to about 20% by weight.

5. The composition of claim 1, wherein the one or more carboxylic acids range from about 150 ppm to 20% by weight.

6. The composition of claim 1, wherein the one or more carboxylic acids range from about 12% to 20% by weight.

7. The composition of claim 1, wherein the one or more carboxylic acids are selected from the group consisting of C₂₋₁₀ fatty acids, dicarboxylic acids, tricarboxylic acids, α-keto carboxylic acids, β-keto carboxylic acids, and mixtures thereof.

8. The composition of claim 1, wherein the one or more carboxylic acids comprise acetic acid ranging from about 150 ppm to about 20% of the total weight of the composition.

9. The composition of claim 1, wherein the one or more carboxylic acids comprise acetic acid ranging from about 15% to about 20% of the total weight of the composition.

10. The composition of claim 8, wherein the one or more carboxylic acids further comprises one or more additional acids selected from the group consisting of propionic acid, citric acid, succinic acid, glutaric acid, adipic acid, suberic acid, malonic acid, lactic acid, glycolic acid, oxalic acid, pyruvic acid, citramalic acid, acetoacetic acid, citraconic acid, maleic acid, and any mixture thereof.

11. The composition of claim 1, wherein the one or more peracids range from about 100 ppm to about 12% of the total weight of the composition.

12. The composition of claim 1, wherein the one or more peracids range from about 6% to about 12% of the total weight of the composition.

13. The composition of claim 1, wherein the peracid is peracetic acid in an amount ranging from about 100 ppm to about 12% of the total weight of the composition.

14. The composition of claim 1, wherein the peracid is peracetic acid in an amount ranging from about 6% to about 12% of the total weight of the composition.

15. The composition of claim 1, wherein:
the hydrogen peroxide ranges from about 5% to about 25% by weight,
the one or more carboxylic acids ranges from about 10% to about 30% by weight, and
the one or more peracids ranges from about 4% to about 20% by weight,
wherein at least one of the peracids has the same number of carbons as one of the carboxylic acids.

16. The composition of claim 1, wherein:
the hydrogen peroxide ranges from about 5% to about 25% by weight,
the carboxylic acid comprises acetic acid in an amount ranging from about 5% to about 22% by weight, and
the peracid comprises peracetic acid in an amount ranging from about 0.25% to about 14% by weight.

17. The composition of claim 1, wherein:
the hydrogen peroxide ranges from about 125 ppm to about 625 ppm,
the one or more carboxylic acids ranges from about 150 ppm to about 750 ppm, and
the one or more peracids ranges from about 100 ppm to about 500 ppm.

18. The composition according to claim 1, wherein the one or more carboxylic acids comprises acetic acid in an amount of from about 375 ppm to about 500 ppm.

19. The composition according to claim 1, wherein the one or more peracids is peracetic acid in an amount of from about 150 ppm to about 350 ppm.

20. The composition according to claim 1, wherein the composition has a peracid concentration of between about 125 ppm and 400 ppm.

21. The composition according to claim 1, wherein
the hydrogen peroxide ranges from about 125 ppm to about 3 wt %,
the one or more carboxylic acids ranges from about 150 ppm to about 3 wt %, and
the one or more peracids ranges from about 100 ppm to about 500 ppm, and
wherein at least one of the peracids has the same number of carbons as one of the carboxylic acids.

22. The composition of claim 1, wherein
the hydrogen peroxide ranges from about 55,000 ppm to about 120,000 ppm,
the one or more carboxylic acids ranges from about 70,000 ppm to about 120,000 ppm, and
the one or more peracids ranges from about 6,500 ppm to about 22,000 ppm.

23. The composition of claim 1, wherein the peracid concentration is between about 100 ppm and about 25,000 ppm.

24. The composition of claim 1, wherein
the hydrogen peroxide ranges from about 5,000 ppm to about 20,000 ppm,
the one or more carboxylic acids ranges from about 5,000 ppm to about 20,000 ppm, and
the one or more peracids ranges from about 100 ppm to about 300 ppm, and
wherein at least one of the peracids has the same number of carbons as one of the carboxylic acids.

25. The composition of claim 1, which is in a form selected from the group consisting of a solution, gel or a foam.

26. (canceled)

27. The composition according to claim 1, further comprising a stabilizer selected from the group consisting of etidronic acid, dipicolinic acid, and dipicolinic acid N-oxide.

28. (canceled)

29. The composition according to claim 1, further comprising an abrasive aid selected from the group consisting of calcium carbonate, a silica gel, silica precipitate, silicate, alumina, phosphate, phosphate salt, hydrated aluminum oxide, magnesium carbonate, and a combination thereof.

30. A method for cleansing dental prosthetics and removable orthodontic devices, comprising contacting the dental prosthetic or removable orthodontic device with the composition of claim 1.

31. The method of claim 30, wherein the composition is contacted with the dental prosthetic or removable orthodontic device by dispensing a pressurized solution of the composition on a surface of the dental prosthetic or removable orthodontic device, or is contacted with the dental prosthetic or removable orthodontic device by soaking the surface of the dental prosthetic or removable orthodontic devices in a solution of the composition.

32. (canceled)

33. The method of claim 30, wherein the composition has a peracid concentration between about 100 ppm and about 25,000 ppm.

34. The method of claim 30, wherein the composition has:
a hydrogen peroxide concentration ranging from about 5,000 ppm to about 20,000 ppm,
a carboxylic acid concentration ranging from about 5,000 ppm to about 20,000 ppm, and
a peracid concentration ranging from about 100 ppm to about 300 ppm.

35. The method of claim 30, wherein the composition has:
a hydrogen peroxide concentration ranging from about 125 ppm to about 20,000 ppm,
a carboxylic acid concentration ranging from about 150 ppm to about 20,000 ppm, and
a peracid concentration ranging from about 100 ppm to about 300 ppm.

36. (canceled)

37. A solid product for cleansing dental prosthetics or removable orthopedic devices, wherein the solid product is formed by removing water from the aqueous composition of claim 1.

38. (canceled)
